(19) United States
(12) Patent Application Publication
(10) Pub. No.: US 2003/0166692 A1
(43) Pub. Date: Sep. 4, 2003

(54) Composition useful as anticancer drug and radiosensitizer

(75) Inventors: Jong-Kuk Park, Seoul (KR); Young-Do Yoo, Seoul (KR)

Correspondence Address: Beverly A. Lyman, Ph.D., Wood, Herron & Evans, L.L.P., 2700 Carew Tower, 441 Vine Street, Cincinnati, OH, 45202-2917, US

(73) Assignee: PHARMGENIA CO., LTD. of Developmental Genetics Laboratory,

(21) Appl. No.: 10/374,561

(22) Filed: Feb. 25, 2003

(30) Foreign Application Priority Data
Feb. 27, 2002 (KR) 2002-10545

(51) Int. Cl. A61K 31/44

(52) U.S. Cl. 514/357

(57) ABSTRACT

The present invention relates to the novel use of the compound [N′-(phenyl-pyridin-2-yl-methylene)-hydrazine carbodithioic acid methyl ester] as an anticancer agent. The present invention also relates to the novel use of the compound [N′-(phenyl-pyridin-2-yl-methylene)-hydrazine carbodithioic acid methyl ester] in combination with radiotherapy for enhancing radiotherapy on cancerous cells or tumors.

### TECHNICAL FIELD

The present invention relates to the novel use of the compound [N′-(phenyl-pyridin-2-yl-methylene)-hydrazine carbodithioic acid methyl ester] as an anticancer agent. The present invention also relates to the novel use of the compound [N′-(phenyl-pyridin-2-yl-methylene)-hydrazine carbodithioic acid methyl ester] in combination with radiotherapy for enhancing radiotherapy on cancerous cells or tumors.

### BACKGROUND ART

Currently, cancer is one of major causes of adult death, but despite extensive research to find a cure for cancer, more than half of patients diagnosed with cancer finally come to die. Recently, widely used cancer treatments include surgical operations, radiotherapy, chemotherapy, and its combination.

Among said treatments, surgical operations are a local treatment having limited effects only on cancerous cells-or tumors-excised sites. Accordingly, surgical operations are not effective in cases where it is impossible to excise cancerous cells or tumors due to their inaccessible location, or in cases where cancerous cells or tumors already spread from an original site to one or more sites (especially, including important organs) in the patient's body.

Radiotherapy is also a local cancer treatment having localized effects only on targeted irradiation sites. In addition, radiotherapy has been reported to be effective in treating only specific kinds of cancers such as lung cancer, mammary cancer, and uterine cancer, while some other kinds of cancers show only partial effects or develop resistance to radiotherapy.

On the contrary, chemotherapy is a treatment having systemic anticancer effects. However, since targeted cancerous cells or tumors generally develop resistance to them from the beginning of chemotherapy or during chemotherapy, a number of chemotherapeutic drugs, which have been authorized until now for using as anticancer agents, might not completely cure cancers by themselves.

Consequently, in order to enhance anticancer treatment, besides surgical operations based on early diagnosis of cancers, there are still high demands to develop effective chemotherapy, radiotherapy and/or combination thereof, especially for cases where surgical operations are impossible and the risk of recurrence is high.

Meanwhile, chemotherapy-radiotherapy combinations are also being currently attempted for treating a variety of cancers, based on the theory that the said two kinds of cancer treatments mediate anticancer effects via different mechanisms and their toxicities do not overlap. Thus the said combination cancer treatments have been expected to have synergistic effect on treating cancers.

However, such combination cancer treatments have been proved to be unsatisfactory. Among anticancer drugs developed until now, it has been reported that only taxol and cisplatin may be used as radiotherapy-enhancing agents (or so called “radio-sensitizer”). However, even though they are rather effective in enhancing radiotherapy, they still have some critical defects, especially causing toxicity to patients.

As described above, the necessary conditions of candidate anticancer drugs that can be also used as radiotherapy-enhancing agents are as follows: (1) enhancing the anticancer effect of radiation therapy; (2) causing no damage to normal cells (especially, adjacent to targeted cancerous cells or tumors); and (3) causing no (at least less) toxicity.

### SUMMARY OF THE INVENTION

The present invention provides a pharmaceutical composition useful for treating cancers (preferably, gastric and/or lung cancer) in mammals (preferably, human), which comprises the therapeutically effective amount of the compound of formula I and a pharmaceutically acceptable carrier:

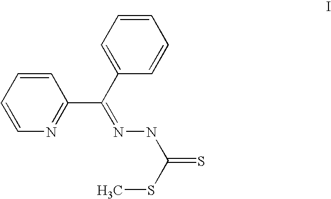

Further, the present invention provides a pharmaceutical composition useful for enhancing radiotherapy on cancers (preferably, gastric and/or lung cancer) in mammals (preferably, human), which comprises the therapeutically effective amount of the compound of formula I and a pharmaceutically acceptable carrier.

Furthermore, the present invention provides a method for treating cancers (preferably, gastric and/or lung cancer) in mammals (preferably, human), which comprises administering the effective amount of the compound of formula I.

Still further, the present invention provides a method for enhancing radiotherapy on cancers (preferably, gastric and/or lung cancer) in mammals (preferably, human), which comprises administering the effective amount of the compound of formula I in combination with radiotherapy. More preferably, the administration of the compound of formula I is sequentially followed by irradiation.

### BRIEF DESCRIPTION OF THE ACCOMPANYING DRAWINGS

FIG. 1 is a graphic diagram showing the survival rate of cancer cells (i.e. human NCI-H460 lung cancer cell line) after the administration of the compound of formula I.

FIG. 2 is a graphic diagram showing the survival rate of cancer cells (i.e. human SNU638 gastric cancer cell line) after the administration of the compound of formula I.

FIG. 3 is a graphic diagram showing the rate of a colony formation of cancer cells (i.e. human NCI-H460 lung cancer cell line) after the administration of the compound of formula I in combination with irradiation. In FIG. 3, the left bar (▪) each shows the rate of a colony formation of irradiated cancer cells in comparison with un-irradiated cancer cells; while the right bar (□) each shows the rate of a colony formation of cancer cells after the compound of formula I (1 ng/ml) had been administered thereto and followed by irradiation in comparison with un-irradiated but the compound of formula I (1 ng/ml)-treated cancer cells.

FIG.4 is a graphic diagram showing the rate of a colony formation of cancer cells (i.e. human A549 lung cancer cell line) after the administration of the compound of formula I in combination with irradiation. In FIG. 4, the left bar (▪) each shows the rate of a colony formation of irradiated cancer cells in comparison with un-irradiated cancer cells; while the right bar (□) each shows the rate of a colony formation of cancer cells after the compound of formula I (0.1 ng/ml) had been administered thereto and followed by irradiation in comparison with un-irradiated but the compound of formula I (0.1 ng/ml)-treated cancer cells.

FIG. 5 is a graphic diagram showing the change of average tumor volume in cancer cells (human NCI-H460 lung cancer cell line) transplanted into mice. In FIG. 5, ●, □, ▴ and ♦ show the change of average tumor volume in mice administered with DMSO (Control); in mice administered only with the compound of formula I (Drug only); in mice only exposed to irradiation (Radiation only); and in mice administered with the compound of formula I and followed by irradiation (Combination), respectively.

### DETAILED DESCRIPTION OF PREFERRED EMBODIMENTS

The present invention provides the use of the compound of formula I [N′-(phenyl-pyridin-2-yl-methylene)-hydrazine carbodithioic acid methyl ester] as an anticancer agent:

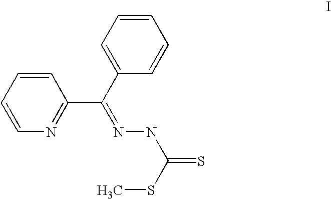

Further, the present invention provides the use of the compound of formula I [N′-(phenyl-pyridin-2-yl-methylene)-hydrazinecarbodithioic acid methyl ester] in combination with radiotherapy for enhancing radiotherapy on cancers:

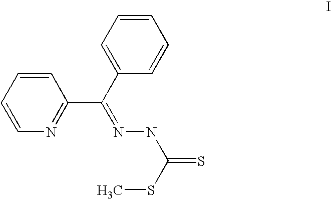

Still further, the present invention provides a pharmaceutical composition useful for treating cancers in mammals (preferably, human), which comprises the therapeutically effective amount of the compound of formula I and a pharmaceutically acceptable carrier.

Furthermore, the present invention provides a method for treating cancers in mammals (preferably, human), which comprises administering the effective amount of the compound of formula I.

Moreover, the present invention provides a method for enhancing radiotherapy on cancers in mammals (preferably, human), which comprises administering the effective amount of the compound of formula I in combination with radiotherapy. In this combination therapy according to the present invention, preferably, administering the compound of formula I is sequentially followed by irradiation.

Still further, the present invention provides the use of the compound of formula I for preparing a medicine useful for treating cancers in mammals (preferably, human).

The compound of formula I according to the present invention is commercially available and its CAS Registration Number is 26158-28-1. The compound of formula I according to the present invention has not been reported or suggested that it has or may have an anticancer activity, whereas its potential as an antifungal agent, a pesticide, a microbicide and an antifouling agent have been reported {see Hossain, M. E. et al, “The preparation, characterization, crystal structure and biological activities of some copper(II) complexes of the 2-benzoylpyridine Schiff bases of S-methyl- and S-benzyldithiocarbazate,” Inorganica Cheminica Acta (1996), 249(2), 207-213; Hossain, M. E. et al, “Synthesis, characterization and biological activities of some nickel(II) complexes of tridentate NNS ligands formed by condensation of 2-acetyl- and 2-benzoylpyridines with S-alkyldithiocarbazates,” Transition Metal Chemistry (1993), 18(5), 497-500; European Patent Application Laid-Open Publication EP0571857 (published on Dec. 1, 1993; Applicant-Bayer A G); German Patent Application Laid-Open Publication DE4207400 (published on Sep. 16, 1993; Applicant-Bayer A G); and European Patent Application Laid-Open Publication EP0778868 (published on Jun. 18, 1997; Applicant-Bayer A G)}.

Under such a circumstance, the inventor has found that the compound of formula I is remarkably effective against cancerous cells or tumors in a host, preferably mammals, and more preferably human. The inventor has also found that the compound of formula I is synergistically effective in treating cancerous cells or tumors when used in combination with radiotherapy, compared to irradiation only.

The phrase “an anticancer agent” in the present application means an agent which is effective in causing the arrest or regression of cancerous cells or tumors in a host. The host to be treated is preferably mammal, and more preferably human.

In an embodiment, this invention provides a pharmaceutical composition comprising the compound of formula I as an active ingredient. Generally, the pharmaceutical compositions additionally comprise a pharmaceutically acceptable carrier diluent, excipient or carrier (collectively referred to herein as carrier materials).

In the pharmaceutical composition of the present invention, the active ingredient will typically be administered in admixture with suitable carrier materials selected with respect to the intended form of administration (i.e. oral tablets, capsules, powders, elixirs, syrups, solutions, suspensions, emulsions and the like).

For example, for oral administration, in the form of tablets or capsules, the active ingredient may be combined with any oral non-toxic pharmaceutically acceptable inert carrier, such as lactose, corn starch, sucrose, cellulose, magnesium stearate, talc, mannitol, ethyl alcohol and the like.

Liquid form preparations include solutions, suspensions and emulsions. For example, D-mannitol, distilled water, p-hydroxybenzoate and the like may be included for parenteral injection solutions.

When desired or needed, suitable binders, lubricants, disintegrants, coloring agents, sweetening agents, flavoring agents, preservatives, buffers, anti-oxidants, coating agents and the like may also be included in the pharmaceutical composition.

Preferably, the compound of formula I is administered orally, intravenously or subcutaneously.

Preferably, the pharmaceutical composition of the present invention is in a unit dosage form. In such form, the pharmaceutical composition may be in a single unit dosage form or be subdivided into suitably sized unit doses containing appropriate quantities of the active ingredient, i.e. an effective amount to achieve the desired purpose of causing the arrest or regression of cancerous cells or tumors in a host.

The amount of the compound of formula I in a unit dose of preparation, whether administered alone or in combination with irradiation, may be widely variable, depending upon a host's age, weight, sex, and severity of the conditions being treated. Preferably, a suitable dose will be in the range of from about 0.5 to about 100 mg/kg of body weight per day.

Chemotherapy treatment plans administering the compound of formula I (such as dosage, duration of treatment, and administration mode) may be specifically made by attending clinicians, depending upon the appropriate course of therapy and a host to be treated.

A broad range of cancers may be treated in accordance with the present invention. These cancers include both primary and metastatic cancers. Specific types of cancers that can be treated include, but are not limited to, gastric cancer, lung cancer, ovarian cancer, prostate cancer, liver cancer, uterine cancer, thyroid cancer, pancreatic cancer, lingual cancer, bile duct cancer, rectal cancer, mammary cancer, skin cancer and other various types of cancer. In a more preferable embodiment, the types of cancer to be effectively treated are gastric and/or lung cancer.

The inventor also discovered that administering the compound of formula I in combination with irradiation is synergistically effective in treating cancerous cells or tumors, in comparison with irradiation only.

Radiation may be administered according to the present invention in a variety of fashions. For example, radiation may be electromagnetic or particulate in nature. Electromagnetic radiation useful in the practice of this invention includes, but is not limited to, x-rays and gamma rays. Particulate radiation useful in the practice of this invention includes, but is not limited to, electron beams, proton beams, neutron beams, alpha particles, and negative pi mesons. Radiation may be delivered using conventional radiological treatment apparatus and methods. Additional information regarding radiation treatments suitable for use in the practice of the present invention may be found in Textbook of Radiation Oncology (see Steven A. Leibel et al., published by W. B. Saunders Company, 1998). Radiation may also be delivered by other methods such as targeted delivery, for example by radioactive “seeds”, or by systemic delivery of targeted radioactive conjugates. Other conventional radiation delivery methods also may be used in the practice of this invention.

The amount of radiation may be variable. In a preferable embodiment, radiation may be administered in amount effective to cause the arrest or regression of cancerous cells or tumors in a host, when the radiation is co-administered with the compound of formula I or a pharmaceutical composition comprising such a compound as an active substance. The radiation may be administered in a variety of treatment plans including the amount and duration of radiation. Choice of the radiation treatment plan may be made by one of skill in the art, depending upon the appropriate course of therapy.

It will be apparent to those skilled in the art that various modifications and variations can be made without departing from the spirit or scope of the invention. Thus, it is intended that various modifications and variations of this invention fall within the scope of the appended claims and their equivalents. Additionally, the following examples are included for the purpose of illustrating the claimed invention, and should not be construed so as to limit the scope of the claimed invention.

### EXAMPLES

### Example 1

In vitro Evaluation of the Compound of Formula I for Anticancer Activity

In this Example, the effect of the compound of formula I as an anticancer agent was evaluated in vitro using human NCI-H460 and SNU638 cancer cell lines.

The inventor purchased the compound of formula I from ChemBridge Corporation (San Diego, Calif.). Human NCI-H460 lung cancer cell line was obtained from ATCC (American Type Culture Collection), while human SNU638 gastric cancer cell line from KCLB (Korean Cell Line Bank).

Under 0.5% CO₂ at 37° C., the human NCI-H460 and SNU638 cancer cell lines were incubated for 4 days in RPMI1640 culture medium (GIBCO/BRL, Grand Island, N.Y.) containing 10% calf fetus serum, penicillin 100 units/ml, and streptomycin 100 μg/ml.

In the case of the NCI-H460 cell line, the compound of formula I was dissolved in DMSO (dimethyl sulfoxide) in order to make its concentration 0.1 ng/ml, 1 ng/ml, 10 ng/ml, 100 ng/ml, 1,000 ng/ml and 10,000 ng/ml, respectively. During incubation, the NCI-H460 cells were treated with each concentration of the compound of formula I, respectively.

In the case of the SNU638 cell line, the compound of formula I was dissolved in DMSO in order to make its concentration 1 ng/ml, 10 ng/ml, 100 ng/ml, 1,000 ng/ml and 10,000 ng/ml, respectively. During incubation, the SNU638 cells were treated with each concentration of the compound of formula I, respectively.

Then, any surviving cancer cells were dyed with MTT [3-(4,5-dimethylthiazol-2-yl)-2,5-diphenyltetrazolium bromide] and quantified at 540 nm with a microplate reader.

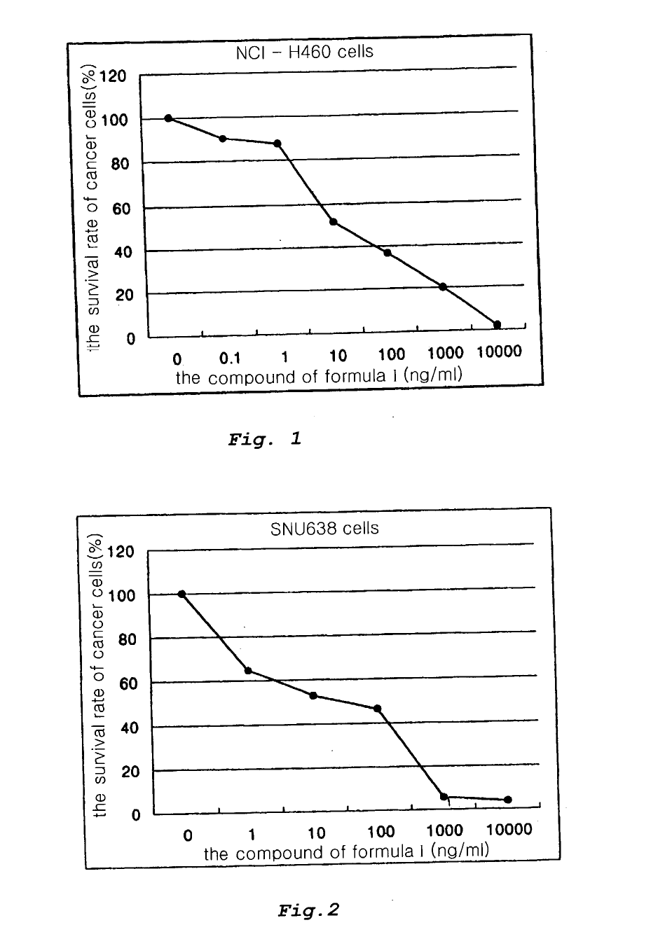

Following Tables 1 and 2, and FIGS. 1 and 2, respectively show the survival rate of cancer cells, based on relative absorbance rate in reference to control (i.e. cancer cells treated with DMSO only). As seen in the Tables 1 and 2, when treated with even quite a small amount (for example, 1 ng/ml) of the compound of formula I, about 12% (in the case of the NCI-H460 cell line) and 35% (in case of the SNU638 cell line) of cancer cells were killed. When treated with 1,000 ng/ml of the compound of formula I, about 80% (in the case of the NCI-H460 cell line) and 94% (in the case of the SNU638 cell line) of cancer cells were killed.

TABLE 1
The survival rate of human NCI-H460 lung cancer cell line

| The compound of formula I | The survival rate of cancer |
|---|---|
| (ng/ml) | cells (%) |
| Control (0) | 100 |
| 0.1 | 90.2 |
| 1 | 87.7 |
| 10 | 51.3 |
| 100 | 36.6 |
| 1,000 | 20 |
| 10,000 | 2 |

TABLE 2
The survival rate of human SNU638 gastric cancer cell line

| The compound of formula I | The survival rate of cancer |
|---|---|
| (ng/ml) | cells (%) |
| Control (0) | 100 |
| 1 | 64.46 |
| 10 | 53.22 |
| 100 | 46.41 |
| 1,000 | 5.71 |
| 10,000 | 3.87 |

### Example 2

In vitro evaluation of the compound of formula I for Radio-Enhanced Anticancer Activity

Using the same method as described in Example 1, human A549 and NCI-H460 lung cancer cell lines (obtained from ATCC) were incubated in RPMI1640 culture medium.

Then, cancer cells were collected and placed into 60 mm (in diameter) petri dishes, 300 cells per petri dish. After the cancer cells were incubated in water-containing CO₂ incubator at 37° C. for 24 hours, 1 ng/ml and 0.1 ng/ml of the compound of formula I dissolved in DMSO were treated to human NCI-H460 and A549 cells, respectively. Four (4) hours later, the cancer cells were irradiated with a different amount of γ-rays using ¹³⁷Cs γ-rays source (Atomic Energy of Canada, Ltd., Canada). After cultivated for 10 days, the colonies were dyed with 1% methylene blue. Colonies having diameters of 200 μm or more were counted using Colony Counter (Imaging Products, Chantilly, Va.).

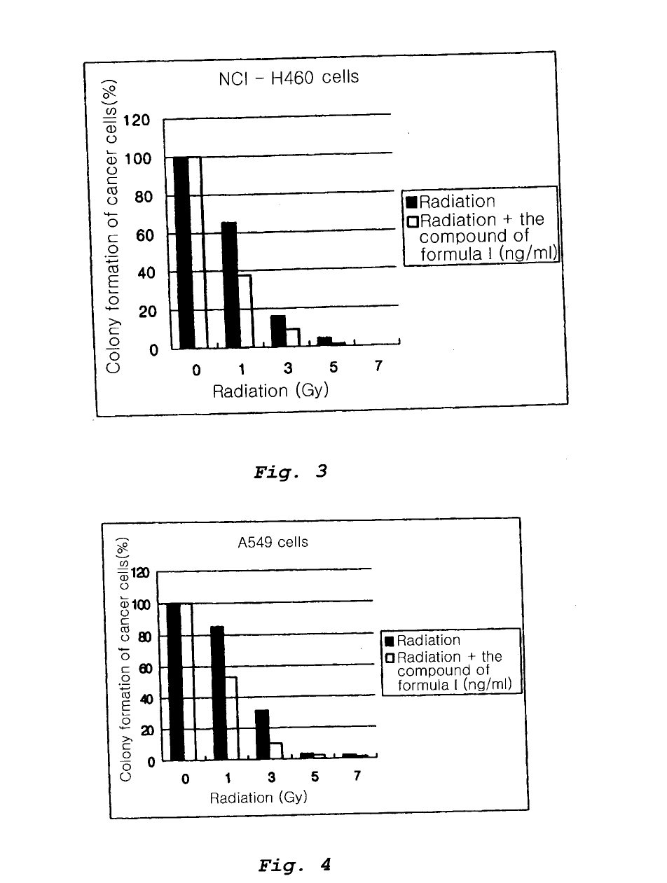

The results regarding colony formation are shown in the following Tables 3 and 4, and FIGS. 3 and 4.

TABLE 3
Colony formation of human NCI-H460 lung cancer cell line

| | The survival rate of | The survival rate of |
|---|---|---|
| | cancer cells | cancer cells |
| Radiation | (only radiotherapy) | (combination therapy) |
| (Gy) | (%) | (%) |
| Control (0) | 100 | 100 |
| 1 | 65 | 37.5 |
| 3 | 16 | 9 |
| 5 | 4 | 1 |
| 7 | 0.04 | 0.01 |

TABLE 4
Colony formation of human A549 lung cancer cell line

| | The survival rate of | The survival rate of |
|---|---|---|
| | cancer cells | cancer cells |
| Radiation | (only radiotherapy) | (combination therapy) |
| (Gy) | (%) | (%) |
| Control (0) | 100 | 100 |
| 1 | 85 | 53 |
| 3 | 31 | 10 |
| 5 | 3 | 2 |
| 7 | 2 | 1 |

As seen in Tables 3 and 4, compared to only irradiation, both irradiation and the administration of the compound of formula I killed more cancer cells. When 1 Gy or more was irradiated, the compound of formula I was shown to be significantly effective in radio-enhanced cancer treatment.

In case of NCI-H460 cell line (see Table 3), 1 Gy of irradiation killed 35% of the cancer cells, in comparison with no irradiation. However, when treated with 1 ng/ml of the compound of formula I and followed by 1 Gy of irradiation, 62.5% of cancer cells were killed, in comparison with the treatment of 1 ng/ml of the compound of formula I and no irradiation. That is, the administration compound of formula I in combination with 1 Gy of irradiation enhanced anticancer effect by 27.5%, in comparison with 1 Gy of irradiation.

In case of A549 cell line (see Table 4), 1 Gy of irradiation killed 15% of the cancer cells, in comparison with no irradiation. However, when treated with 0.1 ng/ml of the compound of formula I and followed by 1 Gy of irradiation, 47% of cancer cells were killed, in comparison with the treatment of 0.1 ng/ml of the compound of formula I and no irradiation. That is, the administration of the compound of formula I in combination with 1 Gy of irradiation increased anticancer effect by 32%, in comparison with 1 Gy of irradiation only.

### Example 3

Animal Model Evaluation of the Compound of Formula I for Anticancer Activity

Six (6) week old mice of BALB/cAnNCrj-nu/nu strain, which were obtained from Chales River Japan Inc. (Japan), were subcutaneously injected lung cancer cells (NCI-H460 cell line, 5×10⁶ cells) to develop tumors.

Mice were selected whose tumor volume reached 120 mm³, and subdivided into 4 groups each having 5 mice: Group 1 (control, treated with DMSO); Group 2 (treated only with 10 mg/kg of the compound of formula I); Group 3 (treated only with 5 Gy of irradiation); and Group 4 (treated with 10 mg/kg of the compound of formula I and 5 Gy of irradiation).

Five (5) times with 5 day intervals between each time, DMSO or the compound of formula I was subcutaneously injected to the mice of Groups 1, 2 and 4, and γ-rays were irradiated to the mice of Groups 3 and 4.

The compound of formula I was dissolved in DMSO to make its concentration 5 mg/ml. 40 μl of DMSO and the compound of formula I each were administered to Groups 1, 2 and 4, respectively. The mice of Group 4 were subcutaneously injected with the compound of formula I, and 4 hours later followed by irradiation. The change of tumor volume in each Group was observed 11 times with 3 to 5 days of interval for 42 days.

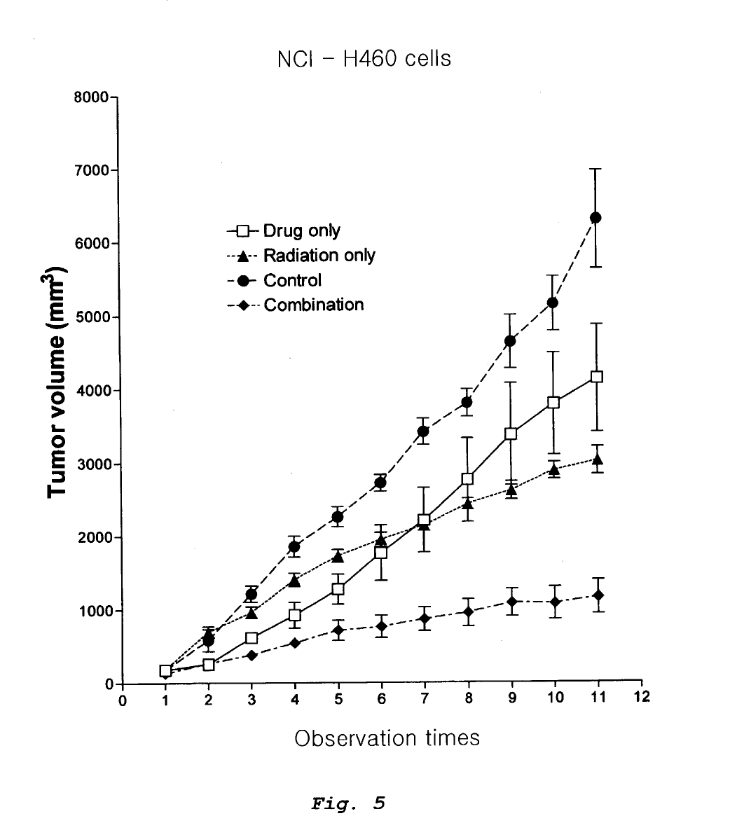

As seen from the following Table 5 and FIG. 5, the increase rate of the tumor volume in Group 2 (the compound of formula I only) was much lower than in Group 1 (control). Furthermore, compared to Group 3 (irradiation only), Group 4 (the compound of formula I+irradiation) showed little increase in tumor volume.

TABLE 5
The change of tumor volume in the NCI-H460 cells
transplanted into mice

| | | The | | |
|---|---|---|---|---|
| Observation | | compound | | |
| times | Control | of formula | Radiation | Combination |
| (days) | (mm³) | I (mm³) | (mm³) | (mm³) |
| 1 (10 days) | 178 | 179 | 188 | 133 |
| 2 (13 days) | 580 | 258 | 693 | 268 |
| 3 (18 days) | 1,216 | 614 | 961 | 379 |
| 4 (21 days) | 1,856 | 923 | 1,405 | 539 |
| 5 (24 days) | 2,262 | 1,274 | 1,725 | 710 |
| 6 (26 days) | 2,720 | 1,766 | 1,948 | 760 |
| 7 (29 days) | 3,418 | 2,211 | 2,142 | 862 |
| 8 (33 days) | 3,805 | 2,759 | 2,433 | 950 |
| 9 (36 days) | 4,629 | 3,374 | 2,612 | 1,086 |
| 10 (39 days) | 5,144 | 3,787 | 2,883 | 1,079 |
| 11 (42 days) | 6,290 | 4,133 | 3,017 | 1,164 |

What is claimed is:

1. A pharmaceutical composition useful for treating cancers in mammals, which comprises the therapeutically effective amount of the compound of formula I and a pharmaceutically acceptable carrier:

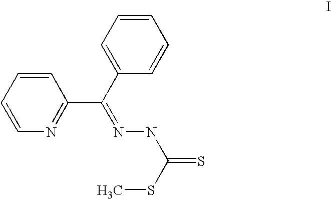

2. The pharmaceutical composition as claimed in claim 1, wherein the mammals are human.

3. The pharmaceutical composition as claimed in claim 1, wherein the cancers are gastric and/or lung cancer.

4. A pharmaceutical composition useful for enhancing radiotherapy on cancers in mammals, which comprises the therapeutically effective amount of the compound of formula I and a pharmaceutically acceptable carrier:

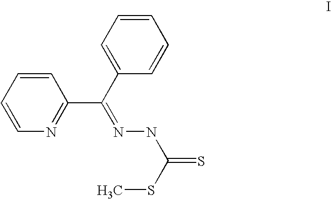

5. The pharmaceutical composition as claimed in claim 4, wherein the mammals are human.

6. The pharmaceutical composition as claimed in claim 4, wherein the cancers are gastric and/or lung cancer.

7. A method for treating cancers in mammals, which comprises administering the effective amount of the compound of formula I:

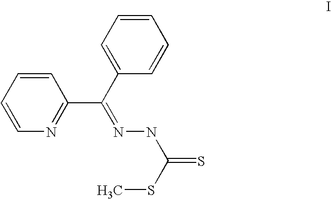

8. The method as claimed in claim 7, wherein the mammals are human.

9. The method as claimed in claim 7, wherein the cancers are gastric and/or lung cancer.

10. A method for enhancing radiotherapy on cancers in mammals, which comprises administering the effective amount of the compound of formula I in combination with radiotherapy:

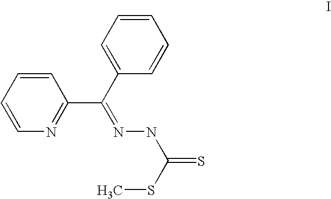

11. The method as claimed in claim 10, wherein administering the compound of formula I is sequentially followed by irradiation.

12. The method as claimed in claim 10 or 11, wherein the mammals are human.

13. The method as claimed in claim 10 or 11, wherein the cancers are gastric and/or lung cancer.
